(19) United States
(12) Patent Application Publication
(10) Pub. No.: US 2010/0112033 A1
(43) Pub. Date: May 6, 2010

(54) STENTS COATED WITH NO- AND S-NITROSOTHIOL-ELUTING HYDROPHLIC POLYMERIC BLENDS

(75) Inventors: Marcelo Ganzarolli de Oliveira, Campinas (BR); Alexander Marra Moreira, Sao Paulo (BR); Amedea Barozzi Seabra, Paulinia (BR); Maíra Martins de Souza Godoy Simões, Campinas (BR); Spero Penha Morato, Osasco (BR)

Correspondence Address: TROUTMAN SANDERS LLP;5200 BANK OF AMERICA PLAZA, 600 PEACHTREE STREET, N.E., SUITE 5200, ATLANTA, GA, 30308-2216, US

(73) Assignees: Universidade Estadual de Campinas UNICAMP, Campinas, SP (BR); SCI-TECH Produtos Medicos LTDA, Goiania, GO (BR)

(21) Appl. No.: 11/997,087

(22) PCT Filed: Apr. 19, 2006
(86) PCT No.: PCT/BR2006/000073

§ 371 Date: Jan. 30, 2009

(30) Foreign Application Priority Data
Jul. 28, 2005 (BR) PI0503201-6

(51) Int. Cl. A61K 33/00; A61F 2/00; A61P 9/00; A61L 33/04; A61F 2/82

(52) U.S. Cl. 424/425; 424/718; 427/2.25; 623/1.46; 623/1.42

(57) ABSTRACT

This invention relates to stents coated with hydrophilic polymers containing S-nitrosothiols, which are able to provide local delivery of both nitric oxide and S-nitrosothiols by diffusion. This device is intended for coronary angioplasty applications with the purpose of inhibiting acute and chronic restenosis and refers to processes of stent coating with hydrophilic polymers containing incorporated S-nitrosothiols. This invention refers to an intracoronary implant device used in medical procedures, and introduces new S nitrosothiol-eluting stents coated with hydrophilic polymer multilayers. The hydrophilic polymers used for coating are polyvinyl alcohol, polyvinylpirrolidone and polyvinyl alcohol/polyvinylpirrolidone, polyvinyl alcohol/polyethylene glycol, polyvinylpirrolidone/polyethylene glycol and polyvinyl alcohol/polyvinylpirrolidone/polyethylene glycol blends. The S-nitrosothiols incorporated to the polymer coatings are mainly primary S-nitrosothiols, characterized by the fact of the nitric oxide (NO) molecule being covalently bound to a sulfur (S) atom which, in turn, is linked to a primary carbon in the molecule's structure, thus constituting the S—NO chemical group. The coating processes include immersion of the stents in polymer solutions containing S-nitrosothiols and nebulization processes of the polymer solutions containing S-nitrosothiols onto the stent surface.

### FIELD OF THE INVENTION

This invention refers to an intracoronary implant device used in medical procedures, and introduces new S-nitrosothiol-eluting stents coated with hydrophilic polymer multilayers.

More specifically, this invention refers to stents coated with hydrophilic polymers containing S-nitrosothiols, which are able to provide local delivery of both nitric oxide and S-nitrosothiols by diffusion. This device is intended for coronary angioplasty applications with the purpose of inhibiting acute and chronic restenosis and refers to processes of stent coating with hydrophilic polymers containing incorporated S-nitrosothiols.

### BACKGROUND OF THE INVENTION

The percutaneous transluminal coronary angioplasty (PTCA) was introduced as a cardiovascular procedure in 1977 (Gruentzig, A. P.; Senning, A.; Siengenthaler, W. E. Nonoperative dilation of coronary-artery stenosis: Percutaneous Transluminal Coronary Angioplasty. N. Engl. J. Med. 301, 61-8-1979) and revolutionized the treatment of myocardial ischemia resulting from occlusion of subepicardial coronary vessels. PTCA technique was initially based on dilation of the occluded vessel segment by inflation of a catheter-delivered balloon, and its two major limitations were acute reocclusion (incident in approximately 5 to 9% of the cases) and late restenosis (occurring in nearly 30 to 50% of the patients) (Carneiro, R. C.; Oliveira, L. G.; Ribeiro, E.; Silva, L. A.; Vasques, R.; Mussa, M.; Carvalho, V.; Pereira, S. S.; Aboud, E.; Neto Auada, M.; Santos, R. G.; Angrisani Neto, S.; Frange, P. J., Angioplastia Coronária: Causas de insucesso, Revista Brasileira de Cardiologia Invasiva, 4, 5-10-1996). Acute reocclusion is eminently a thrombosis process resulting from platelet activation and triggering of blood-clotting cascade. Restenosis, i.e., coronary arterial lumen reocclusion, results in great part from a cicatricial reparative response to the arterial lesion induced by balloon distension (Bohl, K. S.; West, J. L., Nitric oxide-generating polymers reduce platelet adhesion and smooth muscle cell proliferation. Biomaterials 21, 2273-2278-2000).

This healing process has several characteristics of an inflammatory response and the main obstructive element derives from migration and proliferation of cells with smooth muscular phenotype at the lesion site. In addition to the development of this neointimal layer, vascular lumen occlusion is also due to a phenomenon known as vessel remodeling, which consists in the overall readaptation of the vessel outer diameter to reduce the cross-sectional area circumscribed by the external elastic membrane.

A great advance in coronary angioplasty has been achieved with the development of mechanical devices designed to be permanently implanted into a vessel at the site of a blockage and act as a scaffold to provide internal structural “support” to a narrowed coronary artery segment. The so-called intravascular “stents” or coronary endoprostheses were introduced to clinical use in cardiovascular interventions approximately 18 years ago. (Sigwart, U.; Puel, J.; Irkovitch, M.; Joffre, F.; Kappenberger, L., Intravascular stents to prevent occlusion and restenosis after transluminal angioplasty. N. Engl. J. Med. 316, 70-76-1982).

Stents are catheter-delivered expandable, flexible wire mesh tubes implanted into coronary arteries blocked by atherosclerotic processes with the purpose of widening the luminal diameter at the site of the occlusion and preventing future closure. Although endovascular stenting has in great part overcome acute reocclusion, late restenosis is still the greatest post-angioplasty clinical adverse event and occurs in nearly 20% of treated patients (LeBreton, H.; Topol, E.; Plow, E. F., Evidence for a pivotal role of platelets in vascular reocclusion and restenosis. Cardiovasc. Res., 31, 235-236-1996).

In the early 90's, intracoronary stenting accounted for a high incidence of thrombotic complications (10 to 25%) (Serrius, et al., 1991). The first attempt to reduce thrombosis was the administration of systemic anticoagulant drugs. However, this approach led to an increase in the number of vascular complications. Therefore, the focus shifted to the development of stents coated with anti-thrombotic substances that would neutralize the trombogenicity inherent to stent metal surface. The first covered stents were used in 1991 and had a heparin coating. The results obtained with this stent and the findings of subsequent investigations demonstrated a significant reduction in thrombus formation in different animal models (Hardhammar P A, van Beusekom H M, Emanuelsson H U et al. Reduction in thrombotic events with heparin coated Palmaz-Schatz stents in normal porcine coronary arteries. Circulation 1996; 93:423-430). Further studies showed absence of thrombosis in a large number of patients that received coated stent implant devices. The success of the heparin-coated stents served as background for introduction of a new concept of coating the stents with polymeric materials that would serve as matrices for incorporation of several pharmacological agents. In view of the excellent results in thrombosis reduction, the studies were focused on developing strategies to treat restenosis by inhibition of cell proliferation. This approach brought about the idea of providing local delivery of drugs from the stent surface directly to the vessel wall. Therefore, drug-eluting stents were developed with the purpose of providing local release of drugs with anti-inflammatory, anti-proliferative, anti-migratory and pro-endothelial effects. The pharmacological agents elute from the stent surface to which they are incorporated either in their pure form or adhered to polymeric matrices. Currently, there is a great interest in the development of stent coating materials that can provide elution of drugs with these actions, as well as new polymeric matrices that might be used for drug incorporation.

The nitric oxide (NO), which is endogenously synthesized in the mammalians, prevents platelet activation and platelet adherence, reduces the proliferation of smooth muscle cells, stimulates the proliferation of endothelial cells and the genesis of new vessels, and promotes vasodilatation of blood vessels. Therefore, local release of NO from the surface of coated stents has a great potential in thrombosis prevention and might also reduce post-angioplasty restenosis (Mowery, K. A.; Schoenfisch, M. H.; Saavedra, J. E.; Keefer, L. K.; Meyerhoff, M. E., Preparation and characterization of hydrophobic polymeric films that are thromboresistant via nitric oxide release. Biomaterials, 21, 9-21-2000).

Photopolymerizable, polyethylene glycol (PEG)-based hydrogels have been claimed to be capable of releasing NO in physiological medium for long periods of time ranging from hours to months, depending on polymer formulation. Other studies have shown that platelet aggregation and the proliferation of smooth muscle cells in collagen-coated surfaces were inhibited after blood exposure to such NO-eluting hydrogels (Brieger, D.; Topol, E. Local drug delivery systems and prevention of restenosis. Cardiovasc. Res., 35, 405-413-1997).

Other investigations refer to stents coated with polymer and therapeutic agents. Sousa et al. reported that patients submitted to angioplasty with implantation of sirulimus-coated stents in coronary arteries presented minimal neointimal hyperplasia six months after the stenting procedure (Sousa J E, Costa M A, Abizaid A, Abizaid A S, Feres F, Pinto I M, Seixas A C, Staico R, Matos L A, Sousa A G, Falotico R, Jaeger J, Popma J J, & Serruys P. Lack of neointimal proliferation after implantation of sirulimus-coated stents in human coronary arteries. Circulation 2001; 103:192-195). A previous study showed that self-expanding polymer-coated stents implanted in porcine coronary arteries reduced the incidence of thrombosis in 38% compared to uncoated bare metal stents. However, the polymer-coated stents did not reduce neointimal hyperplasia significantly (Van Der Giessen, Van Beusekon H M, Van Houten C D et al.; Coronary stenting with polymer-coated and uncoated self-expanding endoprostheses in pigs. Coron Artery Dis 1992; 3:631-640). Endovascular stents with different NO-eluting coatings have also been investigated and have shown variable effects (Etteson D S, Edelman E R; Local drug delivery: an emerging approach in the treatment of restenosis. Vasc Med. 2000; 5:97-102) e Bertrand O F, Siphenia R, Mongrain R., Rodes J, Tardifi J C, bilodeU I, Cote g, Bourassa M G; Biocompatibility aspects of new stent technology. J Am Coll Cardiol. 1998; 32:562-571).

Nitric oxide-releasing crosslinked polyethyleneimine microspheres with 51-h half-life were incorporated into the pores of coronary grafts to prevent thrombosis and restenosis (Pullfer S K, Ott D, Smith D J; Incorporation of Nitric Oxide-releasing crosslinked polyethyleneimine microspheres into vascular grafts. J Biomed Mat Res. 1997; 37:182-189). Likewise, [N(O)NO] groups were incorporated to polymeric matrices to modulate the NO releasing time and revealed potential antiplatelet activity in endovascular stents (Smith D J, Chakravarthy D, Pullfer S, Simmons M L, Hrabie J A, Citro M L Saavedra J E, Davies K M, Hutsell T c, Mooradian D L, Hanson S R, Keefer L K; Nitric oxide releasing polymers containing [N(O)NO]-group. J Med Chem. 1996; 39:1148-1156). In another study, bovine S-nitrosated albumin applied to damaged vascular site in rabbit coronary artery was proved to reduce stenosis (Marks D S, Vita J S, Folts J D, Keaney J F Jr, Welch G N, Loscalzo J., Inhibitions of neointimal proliferation in rabbits after by a single treatment with a protein adduct of nitric oxide. J. Clin Invest. 1995; 96: 2630-2638). NO release from bovine albumin compared to non-nitrosated polymer reduced platelet aggregation in 50-70% and neointimal formation in 40% (Maalej N, Albrecht R, Loscalzo J, Folts J D, The potent Platelet inhibitory effects of S-nitrosated albumin coating of artificial surfaces. J. Am Coll Cardiol. 1999; 33:1408-1414). Stents coated with fibrin layers have also been investigated. Fibrin has been considered an excellent candidate for controlled drug delivery because it has a slow and prolonged degradation (lasting 1 to 3 months) and can thus completely cover the coronary stented segment (J Am Coll Cardiol. 1998;31(6): 1434-1438). Heparin-impregnated fibrin-coated stents have also been tested and showed an excellent anti-thrombogenic response and lesser neointimal hyperplasia.

Junghan Yoon et al. assessed the effect of a NO-eluting stent on reducing neointimal thickening in a porcine coronary artery injury model by incorporating sodium nitroprusside, a NO donor, into a polyurethane polymer matrix that was coated onto metallic stents (Yoon J, Wu C, Homme J, Tuch R J., Wolff R G., Topol E J, Lincoff M.; Local delivery of nitric oxide from a eluting stent to inhibit neointimal thickening in a porcine coronary injury model. Younsei Medical Journal 2002; 43(2): 242-251). In this study, it was observed that the polymer-coated stent exerted a local biological effect on the arterial wall, with sustained elevation of cyclic guanosine monophosphate (cGMP) level, which indicates a local biological effect of NO. Although local delivery of NO from this device did not reduce neointimal hyperplasia in this porcine model, this polymer-coated stent might be a promising tool for administration of other agents that may modify the reparative tissue responses leading to restenosis. In another study with similar purposes, biodegradable microspheres containing NO donor or biodegradable polymer (polylactide-co-glycolide-polyethylene glycol) were prepared and loaded into channeled stents, showing that stent-based controlled release of a NO donor significantly reduced in-stent restenosis and was associated with an increase in vascular cGMP levels and suppression of proliferation of smooth muscle cells (Do Y S, Kao E Y, Ganaha F, Minamiguchi H, Sugimoto K, Lee J, Elkins C J, Amabile P G, Kuo M D, Wang D S, Waugh J M, Dake M D. In stent restenosis limitation with stent-based controlled-release nitric oxide: Initial results in rabbits. Radiology 2004; 230: 377-382).

The main polymers used as matrixes for drug elution in coated stents are: poly(lactic acid), polyurethane, polytetrafluorethylene, (poly(lactic acid-co-glycolic acid) and polyethylene glycol. Among the NO-donor agents used in studies with drug-eluting stents are sodium nitroprusside, diazeniumdiolates and nitrosoalbumin, which is a nitrosated protein.

Controlled NO elution from stent surface is an attractive therapeutic option for prevention of restenosis since it can allow the delivery of high NO concentrations directly to the lesion site without causing the side effects usually associated with systemic administration of nitric oxide. Considering that post-stenting healing can be a long process, an advantage of NO elution from a polymeric matrix is to provide a long-term release, which widens its inhibitory action on restenosis.

The NO has the capability of binding to certain amino acids containing the sulfhydryl functional group (—SH), which is also denominated as thiol group. This NO binding is known as nitrosation or S-nitrosation and produces an S-nitrosothiol group (RSNO, where R represents the organic molecule to which the SNO group is bound), which, in turn, can release free NO by homolytic cleavage of the S—NO bond (Singh et al., 1999). In mammalians, the formation of nitrosothiols represents a NO transportation and storage mechanism. Several S-nitrosothiols have been found to be endogenously produced in human body, such as S-nitrosocysteine, S-nitrosogluthation and S-nitrosoalbumin, which indicates that other synthetic RSNOs have great chances to act as low-toxicity exogenous sources of nitric oxide. Since the S-nitrosothiols have practically all biochemical functions of free NO, there is currently a great research interest in developing devices that use such substances, or this particular functional group, for providing controlled local delivery of NO with biomedical purposes.

It has been shown that the incorporation of S-nitrosothiols to different polymeric matrixes is feasible, as demonstrated by several BR Patent Applications submitted to the National Institute of Industrial Property (INPI) [No. IP 004232-0; No. IP 0201167-0; No. IP 030784-7 and No. IP PI0401977-6. The No. IP 0201167-0 patent application demonstrates that it is possible to prepare solid polymeric films made from polyvinyl alcohol (PVA) and mixtures of polyvinyl alcohol with polyvinylpirrolidone (PVA-PVP) containing incorporated S-nitrosothiols. These solid matrixes stabilize the S-nitrosothiols and are capable of releasing NO spontaneously from the incorporated S-nitrosothiols when immersed in aqueous medium. Therefore, they have a great potential for use in stent coatings since they can provide nitric oxide delivery to the stented vessel segment, thus reducing the chances for occurrence of in-stent restenosis.

The structural formula of pure polyvinyl alcohol (PVA) is [—CH₂CH(OH)—]ₙ. PVA is a commercially available semicrystalline polymer that has degrees of hydrolysis ranging from 80 to 99%. The structural formula of PVA with degrees of hydrolysis varying from 96 to 80% is [—CH₂CH(OH)—]_{X}[—CH₂CH(O₂CCH₃—]_{Y}. PVA crystallinity is associated with its degree of hydrolysis and influences its solubility and thermal properties. PVA is soluble in highly hydrophilic and polar solvents. The hydroxyl group present in PVA chains promotes the formation of intra and intermolecular hydrogen bonds. PVA is also an excellent adhesive and presents optimal properties as an emulsifying agent due to its low surface tension. PVA is largely used in textile, paper and cosmetic industries.

PVA is a biocompatible polymer that is widely known for its mechanical properties and was one of the first synthetic polymers to be tested in artificial cartilages (Seal et al.; Mater Sci Eng 2001; 34: 147-230). PVA blends may be molded as films and applied as functional materials, including biomedical materials such as dialysis membranes, membranes for replacement of injured tissues, artificial skin, cardiovascular implants and vehicles for controlled delivery of active substances (Cascone et al.; Biomaterials, 1995; 16:569-574 e Giusti et al.; J Mater Sci Mater Méd; 1993; 4: 538-542). The applicability of PVA films as well as films combining PVA with natural polymers, such as collagen, hyaluronan and gelatin (Scotchford et al.; Biomaterials, 1998; 19:1-11) or deoxyribonucleic acid (Aoi et al. Polymer; 2000; 41:2847-2853), has been investigated for medical purposes. In addition, PVA has been extensively used in the pharmaceutical industry for fabrication of tablets and hydrogels containing bioactive drugs (Morita et al.; J Control Rel 2000; 63:297-304).

The structural formula of polyvinylpirrolidone (PVP) is [—CH₂CH(NC₄H₆O)—]ₙ. PVP has a broad applicability and it is used in formulations of detergents, emulsions, suspensions and pigments. In the pharmaceutical industry, PVP is utilized as a vehicle for dissolution and release of drugs in different formulations. Because it is a strong Lewis base, PVP may strongly interact with other molecules by the formation of hydrogen bonds and might act as a proton acceptor. This characteristic is responsible for the miscibility of this polymer with polymers that act as proton donors, such as polyvinyl alcohol.

Polyvinylpirrolidone (PVP) is one of the most commonly used polymers in Medicine due to its water solubility and extremely low toxicity (Higa et al. Radiat Phys Chem 1999; 55:705-707 e Lopes et al.; Biomaterials 2003; 24:1279-1284). Other pharmaceutical applications of PVP include its use as matrix or additive for controlled drug delivery or coprecipitation of other drugs and as a solid dispersion for controlled drug diffusion (Zavos et al.; Contraveption 1997; 56:123-127 e Tantishiyakul et al. Int J Pharm 1999; 181:143-151). Recent studies have described the use of PVP for topical skin application and for transdermal delivery of drugs (Wang et al., J Chem Eng Jpn 2003; 36:92-97). A mixture of polyvinylpirrolidone and polyvinyl alcohol has been used to obtain membranes and fibers for biomedical purposes (Razzak et al., Radiat Phys Chem 1999; 55:153-165 e Cassu et al., Polymer 1997; 38:3908-3911).

Polyethylene glycol (PEG) or polyethylene oxide (PEO) is a non-toxic water-soluble polymer frequently used in the biomedical field. It is commercially available with molar masses ranging from few hundreds to thousands Daltons. The designation PEG is used for low molar mass compounds (below 20,000 g/mol), while the designation PEO is restricted to high molar mass compounds (above 20,000 g/mol). PEGs with molar masses less than 1,000 g/mol are found in the form of stable colorless solutions or pastes. PEGs with high molar masses (above 1,000 g/mol) are available as white powder or flakes. PEG possesses a variety of properties pertinent to biomedical purposes, including insolubility in water at high temperatures and formation of complexes with metallic cations. It also acts as a protein and nucleic acid precipitating agent.

The properties of physically reticulated gels of PVA, PVP and PEG polymers and their blends have been largely investigated. These biocompatible hydrogels have good mechanical properties, can retain a great amount of water, are stable at room temperature and are able to preserve their original shape (Hérnandez et al., Polymer 2004; 46: 5543-5549; Yoshihiro et al. J Mater Sci, 1997; 32: 491-496; Ricciardi et al.; Chem. Mater 2005, 17:1183-1189).

Some S-nitrosothiols are commercialized in their solid form, such as S-nitrosogluthation (GSNO) (ICN Pharmaceutical, Costa Mesa, Calif., USA; Sigma-Aldrich, St. Louis, Mo., USA; Alexis Biochemicals, San Diego, Calif., USA) and S-nitroso-N-acetylpenicillamine (SNAP) (ICN Pharmaceutical, Costa Mesa, Calif., USA; Sigma-Aldrich, St. Louis, Mo., USA; Alexis Biochemicals, San Diego, Calif., USA).

Several methods are currently available for synthesis of S-nitrosothiols in aqueous media. One of these methods consists in the reaction of thiol with sodium nitrate (NaNO₂) in ice bath in an acid medium (HCl). The formed S-nitrosothiols is precipitated by addition of a solvent with polarity lower than that of water, for example, acetone or ether. To avoid the need for addition of another solvent to promote precipitation of S-nitrosothiols, the pH of the solution may be adjusted to 7.4 by adding NaOH base and saline buffer solution (Hart, T. W., Some observations concerning the S-nitroso and S-phenylsulphonyl derivatives of L-cysteine and glutathione. Tetrahedron Letters. 26, 2013-2016, 1985).

U.S. Pat. Nos. 5,593,876, 6,471,347 and 6,124,255 describe methods for thiol nitrosation, namely: 1—Nitrosation by polypeptide exposure to a NO donor under conditions that allow release or transference of nitric oxide from the donor to the polypeptide; 2—Bubbling of a nitric oxide gaseous source through a polypeptide solution during the time required for formation of nitrosothiol (BR Patent Application No. 200100577-A); 3—Exposure of thiols to bovine aortic endothelial cells stimulated for secretion of endothelium-derived relaxing factor (EDRF) by shearing forces; 4—Exposure of thiols to nitric oxide synthetase together with a byproduct and a cofactor; 5—Acidification of the alkaline thiol solutions and species containing nitrite by addition of acid; 6—Synthesis of polynitrosated polyesters from the polyesterification reaction of a diol with a carboxylic dyacid followed by nitrosation of polyester sulphydryls, according to the method described in the BR Patent Application 300.784-7 submitted to the National Institute of Industrial Property (IPI) on Feb. 24, 2003.

The preparation of PVA/PVP polymeric blends, PVA films, PVP films and PVA/PVP blend films containing NO donors has been presented in several studies [Cassu S N, Felisberti M I. Poly(vinyl alcohol) and poly(vinyl pyrrolidone) blends: miscibility, microheterogeneity and free volume change. Polymer 1997; 38:3908-3911, A. B. Seabra, Lilian L. da Rocha, Marcos N. Eberlin, Marcelo G. de Oliveira. Solid films of blended poly(vinyl alcohol)/poly(vinyl pyrrolidone) for topical S-nitrosoglutathione and nitric oxide release Journal of Pharmaceutical Sciences, 2005; Amedea B. Seabra, Gabriela F. P. de Souza, Lilian L. da Rocha, Marcos N. Eberlin, Marcelo Ganzarolli de Oliveira S-Nitrosoglutathione incorporated in poly(ethylene glycol) matrix: potential use for topical nitric oxide delivery” Nitric Oxide, Volume 11, No 3, 2004, P. 263-272], as well as in the BR 200201167, which describes the method for preparation of polymeric blends from PVA/PVP polymers containing S-nitrosothiols as NO donors.

At least 101 patents involving stent coating with polymers and therapeutic agents were registered at the ISI Web of Knowledge Derwent Innovations index databank from 1996 to 2004. Fifty-one patents related to stents and NO donors are registered in the United States Patent and Trademark Office databank. Among these, the following patents stand out: WO 2004017939-A1—Medical devices, especially stents, loaded with a drug “A” intended to inhibit vascular smooth muscle cell proliferation and a drug “B” intended to improve the vascular endothelial cell function. A NO donor, preferably S-nitroso-N-acetylpenicillamine (SNAP) or arginine, is mentioned as drug “B”; WO 2004002367-A1—Drug-eluting stents constituted by several layers applied onto the stent body surface (of which at least two layers are drugs), comprising a polymeric layer, an additive and active ingredients. PVA and PVP are referred as polymers and NO donors are mentioned as antistenotic drugs; US 20040171589-A1 relates to devices and methods for differential and local delivery of NO to the body. The devices have at least two nitric oxide donor compounds with different eluting mechanisms and different half-lives.

To date, the inventions that compose the state of the art in the field of the present invention do not contemplate systems that are capable of eluting, by diffusion, both NO and NO-donor S-nitrosothiols from drug-eluting coated stents to surrounding tissues. They also do not contemplate specifically the use of primary S-nitrosothiols, such as low molar mass amino acids or peptides, which have a great capacity of delivering NO spontaneously, as well as diffusing from hydrosoluble polymeric matrixes to surrounding tissues or aqueous media. In view of this and considering that the primary S-nitrosothiols have the same beneficial effects as those of NO in restenosis inhibition, there is non-fulfilled demand for use of systems that combine local NO delivery with local diffusion of intact NO donors, which are capable of providing prompt transference of NO after their contact or penetration into tissue cells. This demand might, therefore, be fulfilled by the use of these primary S-nitrosothiols incorporated to polymers or mixtures of hydrosoluble polymers.

In addition, the incorporation of S-nitrosothiols in multilayers containing one ore more physically reticulated polymers allows the modulation of NO and S-nitrosothiol delivery without complete coating dissolution. This might lead to more effective outcomes of restenosis inhibition than other ever reported in the literature.

### BRIEF DESCRIPTION OF THE INVENTION

This invention refers to an intracoronary implant device used in medical procedures, and introduces new S-nitrosothiol-eluting stents coated with hydrophilic polymer multilayers.

More specifically, this invention relates to stents coated with hydrophilic polymers containing S-nitrosothiols, which are able to provide local delivery of both nitric oxide and S-nitrosothiols by diffusion. This device is intended for coronary angioplasty applications with the purpose of inhibiting acute and chronic restenosis and refers to processes of stent coating with hydrophilic polymers containing incorporated S-nitrosothiols.

The hydrophilic polymers used for coating are polyvinyl alcohol, polyvinyl pirrolidone and polyvinyl alcohol/polyvinylpirrolidone, polyvinyl alcohol/polyethylene glycol, polyvinylpirrolidone/polyethylene glycol and polyvinyl alcohol/polyvinylpirrolidone/polyethylene glycol blends.

The S-nitrosothiols incorporated to the polymer coatings are mainly primary S-nitrosothiols, characterized by the fact of the nitric oxide (NO) molecule being covalently bound to a sulfur (S) atom which, in turn, is linked to a primary carbon in the molecule's structure, thus constituting the S—NO chemical group.

The coating processes include immersion of the stents in polymer solutions containing S-nitrosothiols and nebulization processes of the polymer solutions containing S-nitrosothiols onto the stent surface.

### DETAILED DESCRIPTION OF THE INVENTION

This invention refers to an intracoronary implant device used in medical procedures, and introduces new S-nitrosothiol-eluting stents coated with hydrophilic polymer multilayers.

More specifically, this invention relates to stents coated with hydrophilic polymers containing S-nitrosothiols, which are able to provide local delivery of both nitric oxide and S-nitrosothiols by diffusion. This device is intended for coronary angioplasty applications with the aim of inhibiting acute and chronic restenosis and refers to processes of stent coating with hydrophilic polymers containing incorporated S-nitrosothiols.

Stent coating is performed with the following hydrophilic polymers: polyvinyl alcohol, polyvinylpirrolidone and polyvinyl alcohol/polyvinylpirrolidone, polyvinyl alcohol/polyethylene glycol, polyvinylpirrolidone/polyethylene glycol and polyvinyl alcohol/polyvinylpirrolidone/polyethylene glycol blends. These polymers might have been submitted or not to reticulation processes. The S-nitrosothiols in the polymer coatings are mainly primary S-nitrosothiols, characterized by the fact of the nitric oxide (NO) molecule being covalently bound to a sulfur (S) atom which, in turn, is linked to a primary carbon in the molecule's structure, hence constituting the S—NO chemical group.

The coating processes include immersion of the stents in polymer solutions containing S-nitrosothiols and nebulization processes of the S-nitrosothiol-containing polymer solutions onto the stent surface.

Even though some Patent Applications for stent coating include the polyvinyl alcohol as a polymer and nitric oxide donors, such as S-nitrosothiols, as the active drug, the invention hereby proposed distinguishes from other inventions because it makes use of PVA/PVP, PVA/PEG or PVA/PEO blends, combined in one or more layers placed onto the stent surface, in addition to the optional crosslinking of the coating polymers by any known or unpublished upcoming jellification process. The utilization of PVA/PVP, PVA/PEG or PVA/PEO blends, as well as the crosslinking of the polymers used for coating allows modulating the plasticity of the polymeric matrixes, making them capable of withstanding the mechanical changes occurring in the stent device due to balloon inflation and expansion during stent deployment. Additionally, the use of such blends allows modulating the eluting velocity of diffusion of both the nitric oxide and the S-nitrosothiols, since the diffusion processes are improved by the greater plasticity of polymer coatings.

Another point that differs this invention from other Patent Applications lies on the fact that the proposed invention uses mainly primary S-nitrosothiols, such as S-nitrosocysteine, S-nitroso-N-acetylcysteine and S-nitrosoglutathione, while other Patent Applications refer primordially to S-nitroso-N-acetylpenicillamine (SNAP), which is a tertiary S-nitrosothiol, or to S-nitrosoalbumin, which is a high molar mass protein. The advantage of using primary S-nitrosothiols for this kind of procedure is that they are found endogenously in the human body and thus present very low toxicity. On the other hand, the S-nitroso-N-acetylpenicillamine (SNAP) is not found endogenously in humans and therefore its administration involves a greater risk of toxicity.

Furthermore, the primary S-nitrosothiols present an extremely intensive biologic activity, stemming from their greater ability of donating nitric oxide to other receptors by both homolytic cleavage of the S—N bond and transnitrosation reactions, in which NO is transferred to other endogenous thiols thereby exerting its biologic action. The greater biologic activity of primary S-nitrosothiols results in a greater thermal instability in aqueous solution. This explains why primary S-nitrosothiols have not been largely used in previous inventions, which have shown a clear preference for S-nitroso-N-acetylpenicillamine (SNAP) due to its greater stability.

The invention hereby presented has also the outstanding quality of providing stabilization of the primary S-nitrosothiols upon their incorporation to polymer matrixes. This is expected to make these compounds commercially viable for the intended purposes because they allow the maintenance of the nitric oxide donor properties, which are important and exert their effects upon diffusion from the donors out of the matrix. If this type of diffusion occurs in direct contact with the tissues, the more intensive biologic action of the primary S-nitrosothiols occurs directly in the tissues towards which these compounds diffuse.

More specifically, the stents to which this invention refers are metallic stents coated with a polymer coating, which are able to provide, by diffusion, local delivery of nitric oxide or at least release of S-nitrosothiol. These stents are loaded on an expansible substrate adapted for implantation in human arteries and veins or vessels of other animals. The polymer coating may comprise one, two, three or more layers. One or more of these layers contain at least one S-nitrosothiol capable of releasing nitric oxide and diffusing into the tissues adjacent to the site device where the stent was implanted. The concentration of each S-nitrosothiol (or the mixture of different S-nitrosothiols) in the polymer layer may range from 0.0001% to 99% in mass.

The detailed presentation of this invention depicted above had both descriptive and illustrative purposes. Moreover, it is important to highlight that this description is not intended to restrict the invention to the form (or applications) presented herein. Therefore, within the scopes of this invention, deviations and modifications that comply with the above explained fundaments and fulfill the requirements of specific skills or technical knowledge are allowed.

The above described modalities are intended to better explain the known ways of using this invention and allow the technical personnel working in this field to employ the invention in such or other modalities and with the required modifications for the specific applications or uses of this invention. It is the intention of this invention to comprehend all of its modifications and variations within the scope of this report and the annexed claims.

1. Intracoronary implant device, comprising a stent coated with a solid hydrophilic polymeric film containing one or more incorporated S-nitrosothiols (RSNOs) in concentrations ranging from approximately 1.0×10⁻⁶% mass to their solubility limits in the matrix, which are capable of providing, by diffusion, local delivery of both nitric oxide and S-nitrosothiols, for applications in coronary angioplasty and treatment of chronic and severe restenosis.

2. Intracoronary implant device according to claim 1, wherein the hydrophilic polymers used for stent coating are poly(vinyl alcohol), poly(vinylpirrolidone), poly(vinyl alcohol)/poly(vinylpirrolidone), poly(vinyl alcohol)/poly(ethylene glycol), poly(vinylpirrolidone)/poly(ethylene glycol) and poly(vinyl alcohol)/poly(vinylpirrolidone)/poly(ethylene glycol) blends.

3. Intracoronary implant device according to claim 1, wherein the S-nitrosothiols (RSNOs) are primary S-nitrosothiols.

4. Intracoronary implant device according to claim 1, wherein the S-nitrosothiols contain nitric oxide (NO) covalently bound to a sulfur atom (S), which, in turn, is bound to a primary carbon atom within the molecule's structure, thereby constituting the S—NO chemical group.

5. Intracoronary implant device according to claim 4, wherein the primary carbon atom is linked to only one vicinal carbon atom and to two hydrogen atoms, namely R—CH₂—S—NO, wherein R is the remainder of the molecule.

6. Intracoronary implant device according to claim 1, wherein the hydrophilic polymers are polyvinyl alcohols (PVAs), including all commercially available PVAs, in all existing molar mass ranges and in all existing ranges of degrees of hydrolysis, represented by the structural formula [—CH₂CH(OH)-]n, where n is the number of —CH₂CH(OH)— repetition units that comprise the polymer chains.

7. Intracoronary implant device according to claim 1, wherein the hydrophilic polymers are poly(vinyl alcohols) (PVAs), poly(vinylpirrolidone) polymers (PVPs), or PVA and PVP blends;
wherein the mass percentage of PVP in PVA may vary freely within the limits of miscibility of one polymer into the other;
wherein the PVAs include partially hydrolyzed PVAs which contain nonhydrolyzed chain segments in their structures according to the structural formula —CH₂CH(O₂CCH₃)—, where the hydroxyl (OH) group is replaced by the acetate group (O₂CCH₃), as well as the totally hydrolyzed PVAs; and
wherein the PVPs include all polymers in all existing molar mass ranges represented by the structural formula [—CH₂CH(NC₄H₆O)—]ₙ.

8. Intracoronary implant device according to claim 1, wherein the hydrophilic polymers are poly(ethylene glycol) (PEGs) or poly(ethylene oxide) (PEOs), including all commercially available polymers in all existing molar mass ranges represented by the structural formula [—CH₂CH₂O-]n, where n is the number of —CH₂CH₂O— repetition units.

9. (canceled)

10. (canceled)

11. Intracoronary implant device according to claim 1, wherein the polymers are subjected to a crosslinking process.

12. Intracoronary implant device according to claim 1, wherein over a first polymeric layer is deposited a second layer of pure non-plasticized PVA with molar mass equal or different from that of the first layer.

13. Intracoronary implant device according to claim 1, wherein over the first polymeric layer is deposited a second layer of PVA plasticized with PEG or PEO.

14. Intracoronary implant device according to claim 12, wherein the second pure non-plasticized PVA layer contains one or more incorporated RSNOs.

15. Intracoronary implant device according to claim 1, wherein primary RSNOs and/or a drug is contained within any of the polymeric layers.

16. Intracoronary implant device according to claim 1, further comprising a tertiary RSNO in addition to the primary RSNOs.

17. Intracoronary implant device coating process, comprising covering a stent with hydrophilic polymers containing incorporated S-nitrosothiols by the following steps:
a. A single immersion of the stent in a polymer solution containing one or more S-nitrosothiols;
b. Sequential immersions of the stent in the same or different polymer solutions;
c. Drying of the coating by any drying technique that avoids decomposition of the polymers and/or the RSNOs; and
d. Assembling the device on expansible balloons adapted for implantation in human arteries or veins.

18. Intracoronary implant device coating process according to claim 17, wherein the immersion steps are performed by single or sequential sprinkling or nebulization of the stents by solutions containing one or more S-nitrosothiols.

19. Intracoronary implant device according to claim 8, wherein the PVA, PEG, or PEO can be partially or totally esterified through the esterification of carboxyl groups of heparin with hydroxyl groups of the polymers.
